(19) United States
(12) Patent Application Publication
(10) Pub. No.: US 2018/0289132 A1
(43) Pub. Date: Oct. 11, 2018

(54) ORAL DEVICE FOR LIP AUGMENTATION

(71) Applicant: Barbizon Ventures Inc., Vancouver (CA)

(72) Inventors: Stephanie Friesen, Vancouver (CA); John Friesen, Vancouver (CA); Matthew Friesen, Vancouver (CA)

(21) Appl. No.: 15/948,161

(22) Filed: Apr. 9, 2018

Related U.S. Application Data
(60) Provisional application No. 62/483,142, filed on Apr. 7, 2017.

(51) Int. Cl. A45D 44/22; A61C 9/00; A61F 2/00

(52) U.S. Cl. CPC A45D 44/22; A61C 9/004; A61B 2017/00792; A61C 9/002; A61F 2/0059

(57) ABSTRACT

There is described an oral device for augmenting a user's lip. The oral device includes an arcuate body formed such that a shape of the arcuate body is a cast of a shape of at least a portion of a gum of a user. The body has a height such that, when the body is positioned against the at least a portion of the gum, the body does not overlap with at least part of a visible portion of at least one of the user's teeth. The oral device may include retaining members for retaining the oral device in the user's mouth. The oral device may be printed using a 3D printer.

### FIELD OF THE DISCLOSURE

The present disclosure relates to an oral device for augmenting a lip of a user, and to methods of forming such a device.

### BACKGROUND TO THE DISCLOSURE

Lip augmentation is a type of cosmetic surgery in which a person's lip or lips are artificially enhanced so as to alter their appearance. As a result of lip augmentation, a person's lips generally appear fuller and younger. The effect of lip augmentation can also cause wrinkles in the vicinity of the lip to appear reduced, or to disappear, by virtue of the skin of the lip being stretched through the augmentation.

The process is typically carried out by injecting a filler material into the lip, such as fat, botulinum toxin (“Botox”), or some other dermal filler. The injection of the filler material is not without risks, however, as the procedure is invasive, generally expensive, and requires careful quality control of the filler. In addition the filler can form unsightly lumps beneath the surface of the skin, and, in order to sustain the effect of lip augmentation, repeated injections are required at regular intervals (typically every four to six months).

There therefore remains a need in the art for improved devices and methods for providing lip augmentation. The present disclosure seeks to address this need in the art.

### SUMMARY OF THE DISCLOSURE

In a first aspect of the disclosure, there is provided an oral device for augmenting a user's lip. The oral device comprises an arcuate body formed such that a shape of the arcuate body is a cast of a shape of at least a portion of a gum of a user. The body has a height such that, when the body is positioned against the at least a portion of the gum, the body does not overlap with at least part of a visible portion of at least one of the user's teeth.

While the shape of the arcuate body is a cast of the shape of the at least a portion of the gum, the arcuate body is not necessarily molded to the at least a portion of the gum. Thus, the arcuate body may have been formed using a non-molding process, such as 3D printing. However, the shape of the arcuate body may be sufficiently complementary to, and may sufficiently mirror a corresponding shape of the at least a portion of the gum, such that the arcuate body may effectively be a geometric cast of the shape of the at least a portion of the user's gum. In other words, a gum-contacting surface of the arcuate body may comprise an impression of a surface of the at least a portion of the user's gum.

A visible portion of a tooth may refer to those parts of a tooth that are above the gumline and generally viewable by a person looking into the mouth of the user. By configuring the height of the body such that it does not overlap with at least part of a visible portion of at least one of the user's teeth, the oral device may not obstruct a person's view of the user's teeth. Thus, in use the oral device may generally be hidden from view. In addition, the oral device may be at least partially transparent, or may have a colour that matches a colour of the user's gums. In one embodiment, the body may have a height such that, when the body is positioned against with the at least a portion of the gum, the body does not overlap with any visible portion of any of the user's teeth.

When the body is positioned against the at least a portion of the user's gum, the body may be positioned such that it is flush against the at least a portion of the user's gum.

The oral device may be formed of any suitable material provided that the material is not detrimental to the health of the user. Various resins, polymers, elastomers, plastics, etc. may be used in the manufacture of the oral device.

The oral device may eliminate the need for invasive procedures such as the injection of fat or other dermal material into the lip.

The body may have a length such that, when the body is positioned against the at least a portion of the gum, the body extends at least from a portion of the user's gum lying over a first bicuspid of the user, to a portion of the user's gum lying over another first bicuspid of the user. The portions of the user's gum may be those portions of the gum that are directly adjacent the user's upper and/or lower bicuspids.

The body may have a height such that, when the body is positioned against the at least a portion of the gum, the body does not overlap with any visible portions of either the user's: upper central incisors, upper lateral incisors, and upper cuspids; or lower central incisors, lower lateral incisors, and lower cuspids.

The body may have a length such that, when the body is positioned against the at least a portion of the gum, the body extends from a portion of the user's gum lying over a second molar of the user, to a portion of the user's gum lying over another second molar of the user. The portions of the user's gum may be those portions of the gum that are directly adjacent the user's upper and/or lower second molars.

The body may have a height such that, when the body is positioned against the at least a portion of the gum, the body does not overlap with any visible portions of any of the user's teeth except the user's upper rearmost teeth or the user's lower rearmost teeth. The body may have a height such that, when the body is positioned against the at least a portion of the gum, the body does not overlap with any visible portions of either the user's: upper incisors, upper cuspids, upper bicuspids, upper first molars, and upper second molars; or lower incisors, lower cuspids, lower bicuspids, lower first molars, and lower second molars.

The gum may comprise a maxillary gum of the user or a mandibular gum of the user.

The body may have a thickness sufficient to cause, when the body is positioned against the at least a portion of the gum, a lip of the user to be pushed outwards.

The oral device may further comprise at least one retaining member extending from the body. Each retaining member may be formed such that a shape of the retaining member is a cast of a shape of one or more respective teeth of the user. Each retaining member may be configured, when the body is positioned against the at least a portion of the gum, to engage with the one or more respective teeth so as to assist in retaining the oral device in the user's mouth.

While the shape of a retaining member is a cast of the shape of one or more teeth of the user, the retaining member is not necessarily molded to the one or more teeth. Thus, the retaining member may have been formed using a non-molding process, such as 3D printing. However, the shape of the retaining member may be sufficiently complementary to, and may sufficiently mirror a corresponding shape of the one or more teeth, such that the retaining member may effectively be a geometric cast of the shape of the one or more teeth. In other words, a teeth-facing surface of the retaining member may comprise an impression of a surface of the one or more teeth.

Each retaining member may have formed therein an impression of the one or more respective teeth of the user. Each retaining member may be configured to receive the one or more respective teeth. In one embodiment, each retaining member is groove-shaped and comprises a grooved portion for receiving the one or more respective teeth.

Each retaining member may be configured to moldably contact a rearmost portion of the one or more respective teeth.

The one or more respective teeth may comprise an upper rearmost tooth or a lower rearmost tooth. Each respective tooth may comprise a wisdom tooth. Each retaining member may be configured, when the body is positioned against the at least a portion of the gum, to wrap at least partially around the one or more respective teeth. Thus, the retaining member may act as a hook to assist in retaining the oral device within the user's mouth. The oral device may comprise two such retaining members. Each retaining member may be molded to the shape of the one or more respective teeth.

The arcuate body may be molded to the shape of the at least a portion of the gum. When correctly positioned, the arcuate body may moldably contact the user's gum. Molded contact may refer to contact between the arcuate body and the at least a portion of the gum in which body is in molded engagement with the at least a portion of the gum. The arcuate body may be specifically molded to the shape of the at least a portion of the gum (for example using a three-dimensional printing process as described in more detail below). Alternatively, the molding of the body to the gum may be passive in nature; for example an arcuate body may be placed in contact with the user's gum and may mold to the shape of the gum by virtue of being retained within the user's mouth over an extended period of time.

The arcuate body may be formed by a three-dimensional printing process. The arcuate body may be formed by a molding process.

In a further aspect of the disclosure, there is provided an oral device for augmenting a user's lip. The oral device comprises an arcuate body having a shape complementary to a shape of at least a portion of a gum of a user. The body has a height such that, when the body is positioned against the at least a portion of the gum, the body does not overlap with at least part of a visible portion of at least one of the user's teeth. The oral device further comprises at least one retaining member extending from the body. Each retaining member is formed such that a shape of the retaining member is a cast of a shape of one or more respective teeth of the user. Each retaining member is configured, when the body is positioned against the at least a portion of the gum, to engage with the one or more respective teeth so as to assist in retaining the oral device in the user's mouth.

In this embodiment, the arcuate body may have a shape that is similar to a shape of the at least a portion of the gum. For example, the arcuate body may be generally arc-shaped. However, a gum-contacting surface of the arcuate body may be substantially flat and need not be a cast of the shape of the at least a portion of the gum. However, in other embodiments, the body may be formed such that the shape of the body is a cast of the shape of the at least a portion of the gum.

Each retaining member may have formed therein an impression of one or more teeth of the user. Each retaining member may be configured to receive the one or more teeth.

The one or more respective teeth may comprise an upper rearmost tooth or a lower rearmost tooth. The one or more respective teeth may comprise a wisdom tooth. Each retaining member may be configured, when the body is positioned against the at least a portion of the gum, to wrap at least partially around the one or more respective teeth. The oral device may comprise two such retaining members.

The body may have a length such that, when the body is positioned against the at least a portion of the gum, the body extends at least from a portion of the user's gum lying over a first bicuspid of the user, to a portion of the user's gum lying over another first bicuspid of the user.

The body may have a height such that, when the body is positioned against the at least a portion of the gum, the body does not overlap with any visible portions of either the user's: upper central incisors, upper lateral incisors, and upper cuspids; or lower central incisors, lower lateral incisors, and lower cuspids.

The body may have a length such that, when the body is positioned against the at least a portion of the gum, the body extends from a portion of the user's gum lying over a second molar of the user, to a portion of the user's gum lying over another second molar of the user.

The body may have a height such that, when the body is positioned against the at least a portion of the gum, the body does not overlap with any visible portions of any of the user's teeth except the user's upper rearmost teeth or the user's lower rearmost teeth. The body may have a height such that, when the body is positioned against the at least a portion of the gum, the body does not overlap with any visible portions of either the user's: upper incisors, upper cuspids, upper bicuspids, upper first molars, and upper second molars; or lower incisors, lower cuspids, lower bicuspids, lower first molars, and lower second molars.

The gum may comprise a maxillary gum of the user or a mandibular gum of the user.

The body may have a thickness sufficient to cause, when the body is positioned against the at least a portion of the gum, a lip of the user to be pushed outwards.

Each retaining member may comprise a grooved portion for receiving therein the one or more respective teeth. The one or more respective teeth may comprise the user's left or right upper teeth, or the user's left or right lower teeth, except the user's upper and lower central incisors, upper and lower lateral incisors, and upper and lower cuspids.

The arcuate body may be molded to the shape of the at least a portion of the gum.

The arcuate body may be formed by a three-dimensional printing process.

The arcuate body may be formed by a molding process.

In a further aspect of the disclosure, there is provided a method of making an oral device according to any of the above-described embodiments. The method comprises printing the oral device using a 3D printer. The method may further comprise obtaining image data of the shape of the at least a portion of the gum. The method may further comprise using the image data with the 3D printer to print the oral device. The step of obtaining the image data may comprise scanning the shape of the at least a portion of the gum, using a scanning device. The step of scanning may comprise scanning a cast of the at least a portion of the gum. The method may further comprise obtaining a mold comprising an impression of the at least a portion of the gum. The method may further comprise forming the cast from the mold.

In a further aspect of the disclosure, there is provided a method of making an oral device according to any of the above-described embodiments. The method comprises heating a mouthguard. The method further comprises biting down on the mouthguard with the user's teeth so as to leave an imprint of the user's teeth in the mouthguard. The method further comprises allowing the imprinted mouthguard to cure. The method further comprises removing one or more portions of the cured mouthguard so as to obtain the arcuate body.

In a further aspect of the disclosure, there is provided a kit of parts for making an oral device according to any of the above-described embodiments. The kit of parts comprises a mouthguard; instructions for molding the mouthguard to the at least a portion of the user's gum; and instructions for removing one or more portions of the molded mouthguard so as to obtain the arcuate body.

In a further aspect of the disclosure, there is provided a computer-readable medium having one or more statements and instructions stored thereon, the one or more statements and instructions being configured, when read by a computer, to cause a 3D printer to print an oral device according to any of the above-described embodiments.

### BRIEF DESCRIPTION OF THE DRAWINGS

Specific embodiments of the disclosure will now be described in conjunction with the accompanying drawings, of which:

FIG. 1 is a perspective view of an oral device in accordance with an embodiment of the disclosure;

FIG. 2 is a first view of the oral device of FIG. 1 moldably contacting a cast of a user's gum;

FIG. 3 is a second view of the oral device of FIG. 1 moldably contacting the cast of a user's gum;

FIG. 4 is a third view of the oral device of FIG. 1 moldably contacting a user's gum;

FIGS. 5A and 5B are perspective views of an oral device according to an embodiment of the disclosure;

FIG. 6 is a flowchart showing a method of making an oral device, according to an embodiment of the disclosure; and

FIG. 7 is an example of a mouthguard.

### DETAILED DESCRIPTION OF SPECIFIC EMBODIMENTS

The present disclosure seeks to provide an oral device for lip augmentation, and methods of making such an oral device. While various embodiments of the disclosure are described below, the disclosure is not limited to these embodiments, and variations of these embodiments may well fall within the scope of the disclosure which is to be limited only by the appended claims.

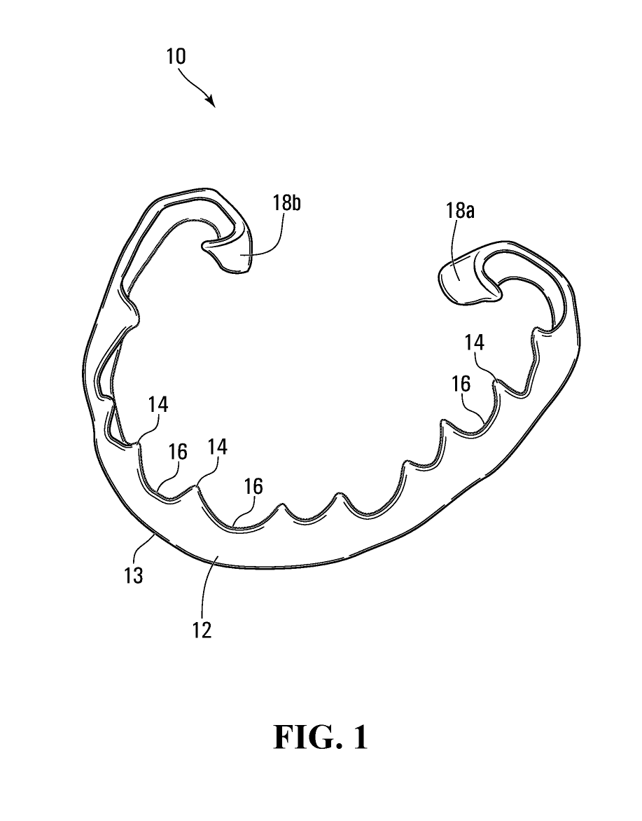

Turning to FIG. 1, there is shown an embodiment of an oral device 10 for augmenting a user's lip. Oral device 10 comprises an arcuate or curved body 12 extending in a roughly elliptical shape. As will be described in more detail below, body 12 is formed such that a shape of body 12 is a cast of a shape of at least a portion of the user's gum. Hereinafter, body 12 is referred to as being molded to a shape of the at least a portion of the user's gum, even though in some embodiments body 12 may have been formed using a non-molding process, such as three-dimensional printing. In the present embodiment, body 12 is molded to a shape of a user's maxillary gum, although in other embodiments body 12 may be molded to a shape of a user's mandibular gum. Body 12 includes a number of indentations comprising alternating peaks 14 and troughs 16 opposed from a base 13.

At either end of body 12, oral device 10 includes a hook-shaped retaining member 18a, 18b. As will be described in more detail below, retaining members 18a, 18b are formed such that a shape of a retaining member is a cast of a shape of a tooth of the user. Hereinafter, retaining members 18a, 18b are referred to as being molded to a shape of respective teeth of the user, even though in some embodiments retaining members 18a, 18b may have been formed using a non-molding process, such as three-dimensional printing. Retaining members 18a, 18b are configured to engage with the respective teeth when oral device 10 is positioned within the user's mouth. Oral device 10 is preferably formed from a suitable plastic, though other materials are contemplated within the scope of this disclosure provided that such materials do not pose a risk to the health of the user.

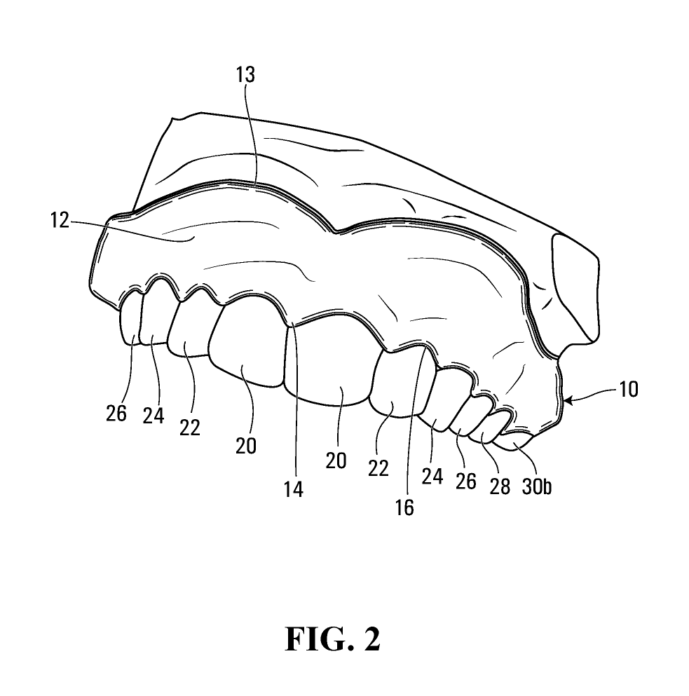

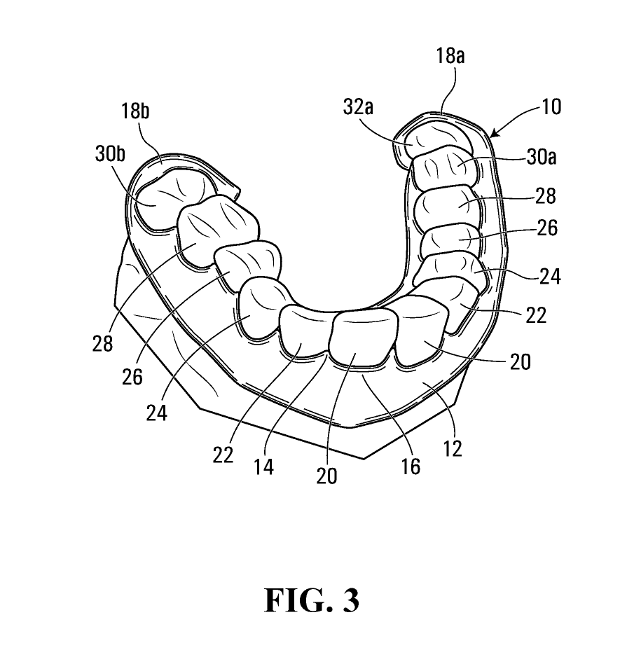

Turning to FIGS. 2 and 3, there are shown different views of oral device 10 positioned in contact with a cast of a user's upper teeth and corresponding gum. In particular, oral device 10 is positioned in flush contact with a cast of the user's maxillary gum (which hereinafter may simply be referred to as the user's maxillary gum). Since body 12 is molded to the shape of the user's maxillary gum, each peak 14 of body 12 is located at the intersection of two adjacent upper teeth. Furthermore, each trough 16 of body 12 follows the gumline of the user's maxillary gum.

With body 12 in molded contact with the user's maxillary gum, each retaining member 18a, 18b engages a rearmost (e.g. posteriormost) tooth of the user's upper teeth. In particular, each retaining member 18a, 18b is shaped so as to wrap around a rearmost one of the user's upper teeth, so as to assist in retaining oral device 10 within the user's mouth. In the present embodiment, retaining member 18a wraps around upper first molar 32a, and retaining member 18b wraps around upper second molar 30b.

Furthermore, as can be seen in FIGS. 2 and 3, the height of body 12 (as defined by troughs 14, peaks 16 and base 13) is configured such that body 12 does not overlap with any visible portion of the user's upper teeth. In the present embodiment, visible is to be interpreted as those portions of the user's teeth than can be seen when one looks into the user's mouth (e.g. portions of the teeth above the gumline and visible to a viewer), and excludes any portions of the user's teeth that are covered by the gums. In alternative embodiments, the height of body 12 (as defined by troughs 14, peaks 16 and base 13) may be configured such that, when body 12 is positioned in flush contact with the user's maxillary gum, body 12 does not overlap with only some of visible portions of the user's upper teeth. In other words, body 12 may have a height such that body 12 extends beyond the maxillary gumline and partially overlaps with one or visible portions of the user's upper teeth.

In the present embodiment, body 12 does not overlap with any visible portions of the upper central incisors 20, upper lateral incisors 22, upper cuspids 24, upper first bicuspids 26, upper second bicuspids 28, and the upper right first molar 30a. However, retaining member 18a partially overlaps a visible portion of upper right second molar 32a, and retaining member 18b partially overlaps a visible portion of upper left first molar 30b. Note that in the present embodiment, body 12 is molded to a maxillary gum of a user with fewer than sixteen upper teeth. In other embodiments, body 12 may be molded to a maxillary gum of a user with any other number of upper teeth (for example the full sixteen teeth expected of an adult in middle age). If for example all sixteen upper teeth are present, then retaining members 18a, 18b are configured to wrap around the user's upper third molars (otherwise known as wisdom teeth). In other embodiments, where the wisdom teeth are absent, retaining members 18a, 18b are configured to wrap around the user's rearmost upper teeth.

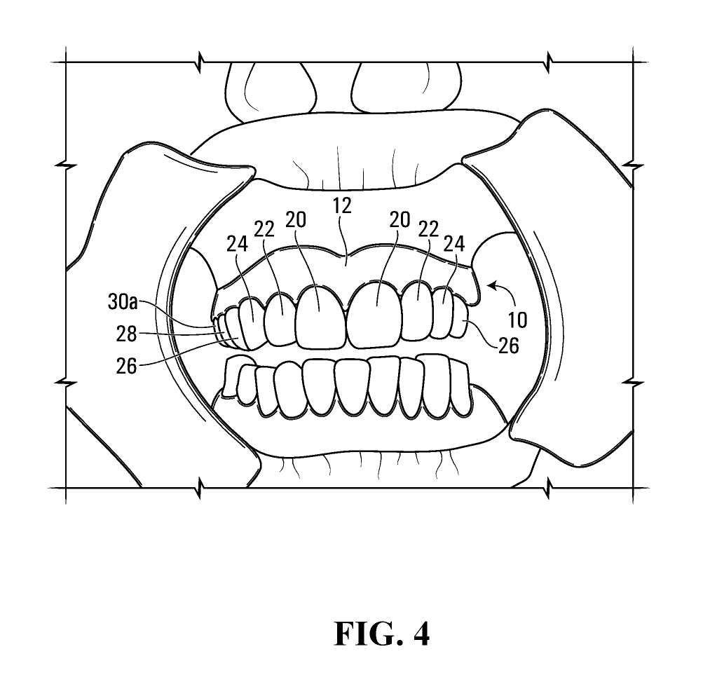

Turning to FIG. 4, there is shown a view of oral device 10 in use, in flush contact or engagement with a user's maxillary gum. As can be clearly seen, body 12 does not overlap with any visible portions of the user's upper teeth. Body 12 generally has a thickness sufficient such that (although not seen in FIG. 4) oral device 12 exerts an outwardly-directed pressure upon the user's upper lip, thereby causing the user's upper lip to be pushed or urged outwardly, resulting in so-called lip augmentation.

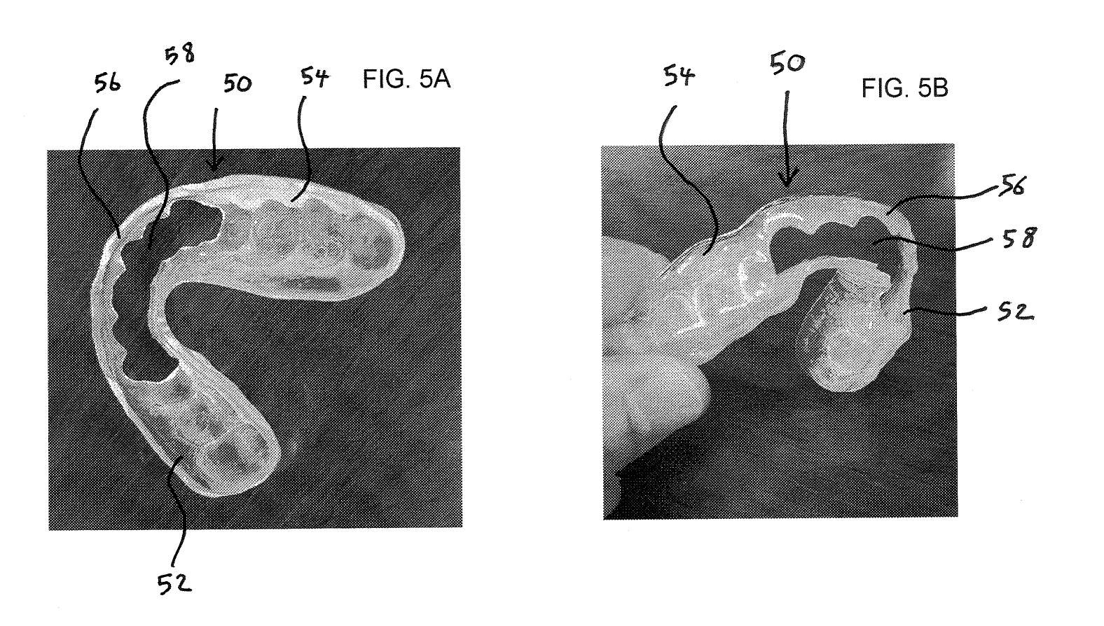

Turning to FIGS. 5A and 5B, there is shown an oral device 50 according to another embodiment of the disclosure. Oral device 50 is similar to oral device 10, except that oral device 50 includes grooved portions 52 and 54 for locating teeth of the user. Each grooved portion 52, 54 has formed therein an impression of several upper teeth of the user. In particular, grooved portion 52 has formed therein an impression of the user's upper left first bicuspid, upper left second bicuspid, and upper left first molar. Grooved portion 54 has formed therein an impression of the user's upper right first bicuspid, upper right second bicuspid, upper right first molar, and upper right second molar. The impressions may have been formed through a molding process in which a mouthguard is heated and bitten into by the user, as described in more detail below. Alternatively, oral device 50 including grooved portions 52 and 54 may have been formed using a three-dimensional printing process, also as described in more detail below.

Oral device 50 includes an aperture 58 defining an arcuate body 56. Body 56 is similar to body 12 in that, when oral device 50 is positioned against the user's gum, body 56 does not overlap with any visible portion of the user's upper front incisors, upper lateral incisors, and upper first bicuspids. Arcuate body 56 is formed such that a shape of body 56 is a cast of a shape of at least a portion of the user's gum. However, in other embodiments, arcuate body 56 has a shape that is merely complementary to a shape of at least a portion of the user's gum, meaning that body 56 may not necessarily be formed such that a shape of body 56 is a cast of a shape of at least a portion of the user's gum. In such a case, a gum-contacting surface of body 56 may be substantially flat, and may not necessarily be molded to a shape of the user's gum.

Grooved portions 52, 54 act as retaining members extending from body 56. Each grooved portion 52, 54 is configured, when body 56 is positioned against the at least a portion of the gum, to engage with upper teeth so as to assist in retaining oral device 50 in the user's mouth. This is but one exemplary embodiment, and as the skilled person would recognise the disclosure embraces many variations of this embodiment, with oral device 50 being configurable such that grooved portions 52, 54 engage with any number of teeth. The more teeth that grooved portions 52, 54 engage with, the more easily oral device 50 is retained in the user's mouth. The fewer teeth that grooved portions 52, 54 engage with, the more hidden oral device 50 will be from a viewer.

While the above-described embodiments have been directed to an oral device for use with a user's maxillary gum, it is entirely possible for two oral devices to be used: one for the user's maxillary gum and another for the user's mandibular gum. Thus, lip augmentation for both of the user's lips may be achieved. The particular features of the two oral devices may differ one from the other, provided nonetheless that both oral devices are configured to achieve the lip augmentation described herein.

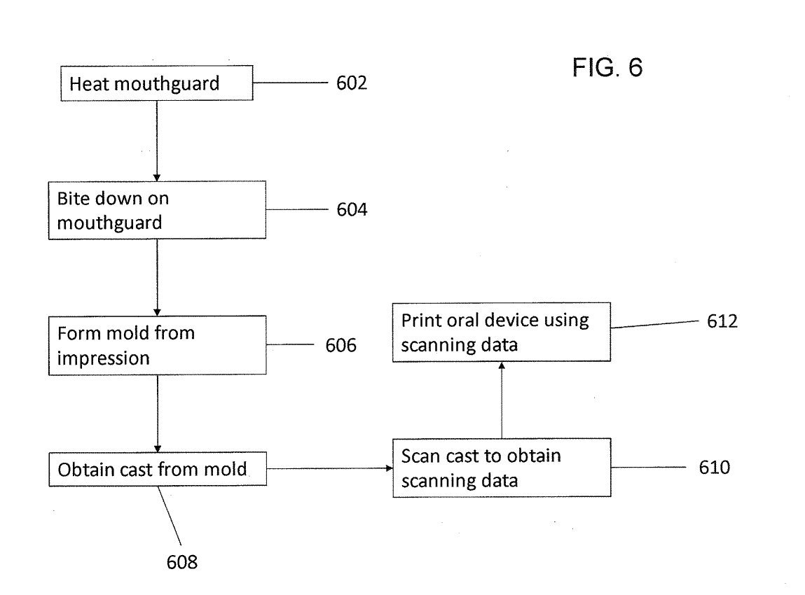

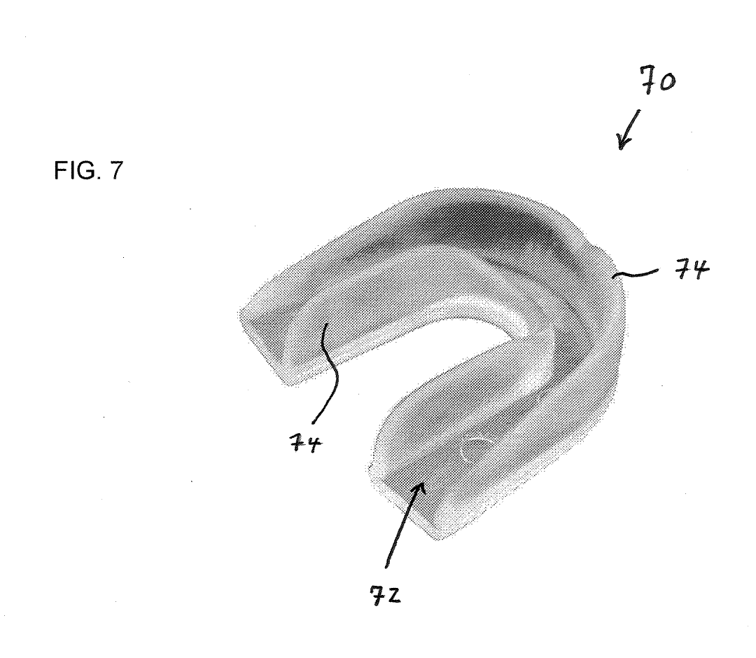

Now turning to FIG. 6, there is shown an exemplary method of forming an oral device as described above. The process begin at step 602 by heating a mouthguard. Mouthguards are well known in the art, and an example of one is shown in FIG. 7. Mouthguard 70 generally comprises a grooved portion 72 delimited by sidewalls 74. In the embodiment of FIG. 7, mouthguard 70 is designed for a user's set of upper teeth. In other embodiments, the mouthguard may comprise a second grooved portion opposite grooved portion 72 and designed to receive the user's set of lower teeth. Mouthguards are typically made of a thermoplastic material or silicone.

Heating the mouthguard softens the material of the mouthguard. At step 604, the user locates their upper teeth within grooved portion 72 and bites down on the mouthguard. The force of the bite leaves an impression or indentation in grooved portion 72, since the material of the mouthguard has been softened at step 702. Once the impression has been formed, the mouthguard is allowed to cure such that the impression becomes permanently formed within mouthguard 70.

Following curing of mouthguard 70, at step 606 a mold of the impressed mouthguard is formed. The process of forming a mold is well known in the art and not discussed in further detail. At step 608, a cast is obtained from the mold. The cast is effectively a negative of the mold. The process of obtaining a cast from a mold is well known in the art and not discussed in further detail. At step 610, the cast is scanned using means known in the art, such as a three-dimensional laser scanner. Scanning of the cast generates scanning data that may be used as an input to a 3D printer. At step 612, the 3D printer is used to print the oral device. Selected portions of the scanning data representing the cast may be selected for 3D printing. In particular, in order to form the above-described oral device, the printer may be instructed to print only that portion of the cast that results in the above-described oral device. In other words, the 3D printer may print an arcuate body that has a height configured such that, when the body is positioned within the user's mouth, the body does not overlap with any of the user's upper teeth.

In an alternative embodiment, it is possible to form the oral device without the use of a 3D printer. In particular, once the cast is obtained, portions of the cast may be manually removed from the cast (for example by cutting or some other form of ablation) so as to leave the arcuate body and retaining members.

Other methods of manufacture that may be used to form the oral device include injection molding, gel casting, slip casting, electroforming, and the use of computer-assisted design/computer-assisted manufacturing (CAD/CAM).

One or more example embodiments have been described by way of illustration only. This description has been presented for purposes of illustration and description, but is not intended to be exhaustive or limited to the form disclosed. Many modifications and variations will be apparent to those of ordinary skill in the art without departing from the scope of the claims. It will be apparent to persons skilled in the art that a number of variations and modifications can be made without departing from the scope of the claims. It is furthermore contemplated that any part of any aspect or embodiment discussed in this specification can be implemented or combined with any part of any other aspect or embodiment discussed in this specification.

1. An oral device for augmenting a user's lip, comprising:
an arcuate body formed such that a shape of the arcuate body is a cast of a shape of at least a portion of a gum of a user, the body having a height such that, when the body is positioned against the at least a portion of the gum, the body does not overlap with at least part of a visible portion of at least one of the user's teeth.

2. The oral device of claim 1, wherein the body has a length such that, when the body is positioned against the at least a portion of the gum, the body extends at least from a portion of the user's gum lying over a first bicuspid of the user, to a portion of the user's gum lying over another first bicuspid of the user.

3. The oral device of claim 2, wherein the body has a height such that, when the body is positioned against the at least a portion of the gum, the body does not overlap with any visible portions of either the user's: upper central incisors, upper lateral incisors, and upper cuspids; or lower central incisors, lower lateral incisors, and lower cuspids.

4. The oral device of claim 1, wherein the body has a length such that, when the body is positioned against the at least a portion of the gum, the body extends from a portion of the user's gum lying over a second molar of the user, to a portion of the user's gum lying over another second molar of the user.

5. The oral device of claim 1, wherein the body has a height such that, when the body is positioned against the at least a portion of the gum, the body does not overlap with any visible portions of any of the user's teeth except the user's upper rearmost teeth or the user's lower rearmost teeth.

6. The oral device of claim 5, wherein the body has a height such that, when the body is positioned against the at least a portion of the gum, the body does not overlap with any visible portions of either the user's: upper incisors, upper cuspids, upper bicuspids, upper first molars, and upper second molars; or lower incisors, lower cuspids, lower bicuspids, lower first molars, and lower second molars.

7. The oral device of claim 1, wherein the body has a thickness sufficient to cause, when the body is positioned against the at least a portion of the gum, a lip of the user to be pushed outwards.

8. The oral device of claim 1, further comprising at least one retaining member extending from the body, each retaining member being formed such that a shape of the retaining member is a cast of a shape of one or more respective teeth of the user, and each retaining member being configured, when the body is positioned against the at least a portion of the gum, to engage with the one or more respective teeth so as to assist in retaining the oral device in the user's mouth.

9. The oral device of claim 8, wherein the one or more teeth comprise an upper rearmost tooth or a lower rearmost tooth.

10. The oral device of claim 8, wherein each retaining member is configured, when the body is positioned against the at least a portion of the gum, to wrap at least partially around the one or more respective teeth.

11. The oral device of claim 8, wherein each retaining member is molded to the shape of the one or more respective teeth.

12. The oral device of claim 1, wherein the arcuate body is molded to the shape of the at least a portion of the gum.

13. A method of making an oral device according to claim 1, comprising:
printing the oral device using a 3D printer.

14. The method of claim 13, further comprising:
obtaining image data of the shape of the at least a portion of the gum; and
using the image data with the 3D printer to print the oral device.

15. The method of claim 14, wherein the step of obtaining the image data comprises scanning the shape of the at least a portion of the gum, using a scanning device.

16. An oral device for augmenting a user's lip, comprising:
an arcuate body having a shape complementary to a shape of at least a portion of a gum of a user, the body having a height such that, when the body is positioned against the at least a portion of the gum, the body does not overlap with at least part of a visible portion of at least one of the user's teeth; and
at least one retaining member extending from the body, each retaining member being formed such that a shape of the retaining member is a cast of a shape of one or more respective teeth of the user, and each retaining member being configured, when the body is positioned against the at least a portion of the gum, to engage with the one or more respective teeth so as to assist in retaining the oral device in the user's mouth.

17. The oral device of claim 16, wherein the one or more respective teeth comprise an upper rearmost tooth or a lower rearmost tooth.

18. The oral device of claim 16, wherein each retaining member is configured, when the body is positioned against the at least a portion of the gum, to wrap at least partially around the one or more respective teeth.

19. The oral device of claim 16, wherein the body has a length such that, when the body is positioned against the at least a portion of the gum, the body extends at least from a portion of the user's gum lying over a first bicuspid of the user, to a portion of the user's gum lying over another first bicuspid of the user.

20. The oral device of claim 16, wherein the body has a height such that, when the body is positioned against the at least a portion of the gum, the body does not overlap with any visible portions of either the user's: upper central incisors, upper lateral incisors, and upper cuspids; or lower central incisors, lower lateral incisors, and lower cuspids.
